(19) United States
(12) Patent Application Publication
(10) Pub. No.: US 2004/0033944 A1
(43) Pub. Date: Feb. 19, 2004

(54) Method of treating erectile dysfunction

(76) Inventors: Craig F. Donatucci, Durham, NC (US); Julie M. Miller, Clarksville, MD (US); Brian H. Annex, Durham, NC (US)

Correspondence Address: NIXON & VANDERHYE, PC, 1100 N GLEBE ROAD, 8TH FLOOR, ARLINGTON, VA, 22201-4714, US

(21) Appl. No.: 10/315,248

(22) Filed: Dec. 10, 2002

Related U.S. Application Data
(63) Continuation of application No. 09/675,659, filed on Sep. 29, 2000, now abandoned.
(60) Provisional application No. 60/157,053, filed on Oct. 1, 1999.

(51) Int. Cl. A61K 38/18

(52) U.S. Cl. 514/12

(57) ABSTRACT

The present invention relates, in general, to erectile dysfunction and, in particular to a method of treating or preventing dysfunction of penile, clitoral or vaginal erectile tissue by administering an angiogenic growth factor, such as vascular endothelial growth factor (VEGF), or active fragment thereof or mimetic thereof.

This application claims priority from Provisional Application No. 60/157,053. the entire content of which is incorporated herein by reference.

### TECHNICAL FIELD

The present invention relate, in general, to erectile dysfunction and, in particular, to a method of treating or preventing dysfunction of penile, clitoral or vaginal erectile tissue by administering an angiogenic growth factor, such as vascular endothelial growth factor (VEGF), or active fragment thereof or mimetic thereof.

### BACKGROUND

Estimates suggest that 25 million US males, or 52% of the men aged 40 to 70 years old, will develop some form of erectile dysfunction by the year 2005 (Feldman et al. J Urol 151: 54-61 (1994), Melman et al, J Urol 161: 5-11 ( 1999)). The introduction of sildenafil citrate (Viagra™, Pfizer Corp) in 1998 greatly expanded the population of affected patients seeking treatment for this disorder. Unfortunately as with all other currently available medications for erectile dysfunction, sildenafil compensates for the dysfunction at the time of use, but does not correct the underlying pathophysiological process. These treatments are most effective in men with mild to moderate erectile dysfunction, and likewise a significant percentage of men are unable to use them successfully.

The physiologic process responsible for penile erection involves corpora cavernosal smooth muscle relaxation, increased arterial inflow and venous occlusion. Nitric oxide (NO), released as a gaseous messenger molecule from endothelial cells and from efferent neurons as a result of erectogenic stimuli, has been identified as the principle mediator of erectile function (Burnett et al, Science 257:401-3 ( 1992)). NO enables relaxation of penile cavernosal trabecular smooth muscle through the generation of cyclic guanosine monophosphate (cGMP) and the subsequent activation of protein kinases, resulting in the phosphorylation of proteins regulating smooth muscle tone (Burnett et al, Science 257:401-3 (1992), Kim et al, J Clin Invest 91:437-42 (1993), Melman et al, J Urol 161: 5-11 (1999)). The principle mechanical event producing penile erection is venous-occlusion (Fournier et at, J Urol 137:163-167 (1987)). With adequate arterial inflow the relaxed corpora cavernosa expand, thereby compressing the subtunical venules against the surrounding fibrous tunica albuginia, trapping blood within the penis and resulting in erection.

A deficiency in smooth muscle function, and commonly a decrease in the quantity of trabecular smooth muscle, is associated with erectile dysfunction both in men and in animal models (Azadzoi et al. J Urol 157:1011-7 (1997), Nehra et al, J Urol 159:2229-2236 (1998), Sattar et al. J Urol 155: 909-912(1996), Wespes et al, J Urol 148:1015-1017 (1991), Wespes et al, J Urol 157:1678-1680 (1997)). Hyperlipidemia is an important risk factor for developing erectile dysfunction in men, and animal models of erectile dysfunction often utilize experimental hyperlipidemia (Azadzoi et al, J Urol 157:1011-7 (1997), Azadzoi et al, J Urol 146:238-40 (1991), Goldstein et al, N Engl J Med 338:1397-404 (1998), Hariawala et al. J Surg Res 63:77-82 (1996), Hood et al, Am J Physiol 274: H1054-8 (1998), Kim et al, J Urol 151:198-205 (1994), Nehra et al, J Urol 159:2229-2236 (1998)). In men, every mmol/liter increase in total cholesterol results in a 1.32 increase in the risk of erectile dysfunction (Wei et al, Am J Epidemiol 140:930-7 (1994)). The hypercholesterolemic rabbit model of erectile dysfunction, first described in 1991. was further characterized as a reproducible method for studying erectile dysfunction (Azadzoi et al, J Urol 146:238-40 (1991), Kim et al, J Urol 151:198-205 (1994)). In this model, endothelium-dependant (acetylcholine-mediated) and endothelium-independent (sodium nitroprusside-mediated) corporal smooth muscle dysfunction develops after 8 weeks on a 1% cholesterol diet. Previous studies (Kim et al, J Urol 151:198-205 (1994)) have demonstrated morphological changes in erectile tissue subjected to hypercholesterolemia, including focal areas of endothelial cell disruption, vacuolated endothelial cells and an increase in lipid-laden vesicles within the smooth muscle cells. Others (Nehra et al, J Urol 159:2229-2236 (1998)) have shown that after 16 weeks on a 0.5% cholesterol diet, the percentage of rabbit corporal smooth muscle cells significantly decrease from 45.4% to 39.2%, similar to the decrease seen in men with veno-occlusive erectile dysfunction. Rabbit and human erectile systems are structurally and functionally similar, and rabbit models are commonly used in the evaluation of pharmacologic treatments for erectile dysfunction (Nehra et al, J Urol 159:2229-2236 (1998)).

Vascular endothelial growth factor (VEGF) is an endothelial cell-specific mitogen in vitro and an angiogenic growth factor in vitro (Hood et al, Am J Physiol 274: H1054-8 (1998), Namikli et al, J Biol Chem 270:31189-95 (1995), van der Zee et al, Circulation 95: 1030-7 (1997), Wu et al, Am J Physiol 271:H2735-9 (1996). Ziche et al, J Clin Invest 99:2625-34 (1997)). Produced by a variety of cells including vascular smooth muscle cells, endothelial cells and inflammatory cells, VEGF has direct effects on both vascular endothelial cells and smooth muscle cells through the activity of receptor tyrosine kinases (Wang et al, Circ Res 83:832-840 (1998), Wang et al, Circ Res 83:832-840 (1998)). Given therapeutically, it has been shown to significantly improve blood flow in vivo in chronic ischemic disorders including ischemic heart and limb models (Hariawala et al,. J Surg Res 63:77-82 (1996), Hood et al. Am J Physiol 274: H1054-8 (1998), Namiki et al, J Biol Chem 270:31189-95 (1995), Takeshita et al, J Clin Invest 93:662-70 (1994)). Multicenter trials are underway assessing the efficacy of VEGF therapy in patients with end-stage coronary artery disease. However, the use of angiogenic growth factors in erectile dysfunction has not been previously explored.

The present invention provides a treatment for dysfunction of penile, clitoral or vaginal erectile tissue that involves the use of an angiogenic growth factor or active fragment thereof or mimetic thereof.

### SUMMARY OF THE INVENTION

The present invention provides a method of preventing or treating dysfunction of penile, clitoral or vaginal erectile tissue. The method comprises administering to a patient in need thereof an amount of an angiogenic growth factor, or active fragment thereof or mimetic thereof, sufficient to effect the prevention or treatment.

Objects and advantages of the present invention will be clear from the description that follows.

### BRIEF DESCRIPTION OF THE DRAWINGS

FIGS. 1A and 1B. Isometric tension studies after VEGF therapy. (FIG. 1A) Endothelium-dependent smooth muscle relaxation was not affected by VEGF treatment. (FIG. 1B). NO-mediated, direct smooth muscle relaxation was improved in cholesterol-fed, VEGF-treated rabbits, with statistical significance at ED75 (P=0.046) and at maximal relaxation (P=0.015). (♦=cholesterol-fed, VEGF-treated; ▪=cholesterol-fed, vehicle treated; ¤=normal diet, vehicle treated)

FIG. 2. Quantification of trabecular smooth muscle content. The smooth muscle content measured by image analysis in normal diet, vehicle-treated animals was assigned a value of 1.0 arbitrary units (mean±SEM) and other treatment groups were assessed relative to this value. Cholesterol-fed, vehicle-treated animals demonstrate significantly decreased overall smooth muscle content compared to normal diet controls (0.86±0.016 arbitrary units, 1±0.022 arbitrary units, P=0.008). Smooth muscle content did not differ among cholesterol-fed rabbits between vehicle or VEGF-treated groups (0.86±0.016 arbitrary units, 0.82±0.016 arbitrary units, P=0.450).

FIG. 3. VEGF immunoexpression. Immunohistochemical VEGF protein expression was determined for each treatment group. Cholesterol-fed, vehicle-treated animals demonstrate significantly decreased VEGF immunoexpression compared to normal diet controls (11.07±1.44 arbitrary units, 24.93±1.09 arbitrary units, P<0.001). VEGF treatment augmented the VEGF expression compared to vehicle controls in both the normal diet animals (37.6±1.12 arbitrary units, 24.93±1.09 arbitrary units, P<0.001) and the cholesterol-fed animals (19.67±1.38 arbitrary units, 11.07±1.44 arbitrary units, P<0.001).

FIGS. 4A and 4B. VEGF treatment significantly augmented endothelium dependent (ACH-mediated) (FIG. 4A) and endothelium independent (SNP-mediated) (FIG. 4B) maximal corporal smooth muscle relaxation.

FIG. 5. VEGF reverses the smooth muscle dysfunction in the hypercholesterolemic rabbit model of erectile dysfunction. NS=Normal saline.

### DETAILED DESCRIPTION OF THE INVENTION

The present invention provides a method of preventing or treating dysfunction of penile, clitoral or vaginal erectile tissue. The method comprises administering to a patient in need thereof an effective amount of an angiogenic growth factor, or active fragment thereof or mimetic thereof. In a specific embodiment, the present invention relates to a method of relieving erectile dysfunction in a male. This method comprises administering to the male an erectile impotence relieving amount of an angiogenic growth factor, or active fragment thereof or mimetic thereof.

Angiogenic growth factors suitable for use in the invention include VEGF and basic fibroblast growth factor (FGF), or active fragments thereof or mimetics thereof. The angiogenic growth factors or active fragments thereof or mimetics thereof, can be used alone or in combination with other agents that enhance the angiogenic growth factor activity. Examples of such activity enhancing agents include endothelial growth factors, such as angiopoietin I.

The invention encompasses any direct or indirect method of administration of the angiogenic growth factor, or active fragment thereof or mimetic thereof, so long as the method is effective in relieving or preventing erectile dysfunction. Preferably, administration is intravenously (e.g., by injection or air gun), however, any method that directs the angiogenic growth factor or active fragment thereof of mimetic thereof to the critical tissue can be used.

For intravenous administration (e.g., intracavernoasl), solutions of the angiogenic growth factor or active fragment thereof or mimetic thereof in a pharmaceutically acceptable carrier (e.g., saline: see also Yang et al, J. Pharm. Exp. Therap. 284:103 (1998)) can be used. The solutions should be sterile. The injection can be made, for example, by needle or air gun. The injection can be made into the corpus cavernosum. Any injection that is effective in relieving impotence can be used.

The amount of angiogenic growth factor, or active fragment thereof or mimetic thereof, administered is that effective in preventing or relieving dysfunction of penile, clitoral or vaginal erectile tissue. For example, the amount administered can be in the range of 10 μg/kg body weight to 250 ρg/kg body weight of, for example, VEGF. The frequency of delivery relates to the frequency of relief needed. Optimum dosage regimens can readily be determined by one skilled in the art and will vary with the agent, the patient and the effect sought.

While administration is described above primarily with reference to intravenous injection of the angiogenic growth factor or active fragment thereof or mimetic thereof, the invention includes within its scope an) of a variety of approaches (direct and indirect) so long as the approach directs the angiogenic growth factor, or active fragment thereof or mimetic thereof, to the critical tissues and thereby relieves or prevents erectile dysfunction. Approaches previously described in connection with myocardial angiogenesis can be adapted for use in the context of the present invention (see, for example, Losordo et al, Am. Heart J. 138 (2 Pt 2):132 (1999); Isner. Am. J. Cardiol. 82 (10A):638 (1998); Henry, BM J 318 (7197):1536 (1999); Isner, Hosp. Prac. 34(6):69-74, 76, 79-80 (1999); Rosengart et al. J. Cardiov. Risk 6(1):29 (1999); Isner et al. J. Clin. Invest. 103(9):1231 (1999): Hyder et al, Mol. Endocrin. 13(6):806 (1999); Veikkola et al, Semin. Can. Biol. 9(3):211 (1999)).

Certain embodiments of the present invention are described in greater detail in the non-limiting Examples that follow.

### EXAMPLE 1

VEGF Restores Corporal Smooth Muscle Response to NO

Experimental Details

Animals: Male New Zealand White rabbits weighing 2.5-3 kg were fed either a normal rabbit diet (n=12) or a 1% cholesterol diet (n=12) (Harland Teklab, Madison, Wis.) for a total of 7.5 weeks. They received an inracavernosal injection of either 0.9 mg VEGF or an equivalent amount of VEGF-vehicle at week 6. Ten days after the injection, the rabbits were euthanized and underwent penectomy with meticulous dissection of the corpora cavernosa from the tunica albuginia. Total serum cholesterol was determined using the enzymatic method prior to the initiation of the experimental diet and on serum samples drawn immediately prior to the procedure. Animal care and handling complied with published Guidelines (Institute of Laboratory Animal Resources, Commission on Life Sciences, National Research Council. Guide for the Care and Use of Laboraiton Animals. Washington: National Academy Press (1996)).

Isometric tension studies. Two corporal strips from each animal were suspended in 5 ml capacity organ baths containing Krebs physiological salt solution (122 mmol/liter NaCl. 4.7 mmol/liter KCl, 1.2 mmol/liter MgCl₂, 2.5 mmol/liter CaCl₂, 15.4 mmol/liter NaHCO₃, 1.2 mmol/liter KH₂PO₄, and 5.5 mmol/liter glucose) maintained at 37° C. and oxygenated with 95% O₂ and 5% CO₂. Strips were attached to an adjustable force transducer and isometric responses were recorded on a multi-channel polygraph (Myograph F-60; Physiograph MK111-S; Narco Bio-Systems, Houston, Tex.). After equilibration at 0.5 grams, optimal preload tension was determined by contracting strips with 60 mmol KCl Krebs solution (60 mmol/liter NaCl, 1.2 mmol/liter MgCl₂, 2.5 mmol/liter CaCl,, 15.4 mmol/liter NaHCO₃, 1.2 mmol/liter KH₂PO₄, and 5.5 mmol/liter glucose) at incrementally increasing levels of preload, until further increase in tension failed to generate an increase in active tension (total tension minus resting tension) of at least 10%. All subsequent testing was then performed at the optimal resting tension for each strip. Strips were sub-maximally pre-contracted with 10⁻⁵ M norepinephrine, and after a contractile plateau was reached, acetylcholine (10⁻⁸ to 10⁻³ M) or sodium nitroprusside (10⁻⁸ to 10⁻⁴ M) was added cumulatively in logarithmic increments. Relaxation in response to each agent is expressed as a percentage of the active tension generated by the 10⁻⁵ M dose of norepinephrine and converted to percentage of maximal response at each dose. These values were plotted against the negative logarithm of the agonist dose to produce relaxation dose-response curves. Logistic regression analysis with logit transformation was performed on the cumulative dose response curves from each treatment group to determine the ED25. ED50 and ED75 for each agent (Finney, Statistical Method in Biological Assay (3 ed.). London: Charles Griffin and Company LTD, p. 349-369 (1978), Kim et al, J Urol 151:198-205 (1994)).

Quantification of trabecular smooth muscle content. Sections of corporal tissue immediately distal to the strips used for isometric tension studies were fixed in 10% formalyn, paraffin-embedded, and stained with the Masson Trichrome stain. Ten randomly selected 40× fields per animal from each treatment group were analyzed using an image analysis system (Olympus IX70 inverted microscope, Optronics DEI-750 image-capturing hardware; PowerTowerPro 180 CPU; Adobe Premiere software) and overall smooth muscle area (red staining) was quantified using NIH Image software (Channon et al, Circulation 98:1905-1911 (1998)). The smooth muscle content measured in normal diet, vehicle-treated rabbits was assigned a value of 1.0 arbitrary units (mean±SEM), and other treatment groups were assessed relative to this value.

Immunohistochemical evaluation of VEGF protein expression. Corporal sections were cryopreserved in 30% sucrose and snap frozen in liquid nitrogen. Frozen sections (5 μm) were made in a cryostat, allowed to return to room temperature, and then fixed in ice-cold acetone for 10 minutes and washed in phosphate-buffered saline solution. After blocking solution (10% horse serum in phosphate-buffered saline), monoclonal horse anti-human VEGF antibody was applied for one hour (Sigma, St. Louis, Mo.). This was followed by sequential incubation with biotinylated secondary antibody, the ABC reagent and the alkaline phosphatase substrate kit (Vector Labs, Burlingame, Calif.). Sections were counterstained with hematoxylin and dehydrated and mounted with Cytosel (Fisher Scientific, Pittsburgh, Pa.), leaving the antigen red. Six randomly selected 40× fields per animal from each treatment group were assessed, and areas of VEGF expression were counted by a single observer.

Statistical evaluation. Each treatment group contained 6 animals (corporal strip n=12). Data are expressed as the mean±standard error of the mean (SEM). The responses of strips from each treatment group to acetylcholine or sodium nitroprusside were compared using the independent samples T-Test. Prior to comparison, samples were checked for normality and no significant deviations were found. The ED50 (sensitivity) and the maximal response were the principal outcomes assessed for each agent, and adjusting for multiple comparisons each outcome was assessed at the 0.025 level. Secondary outcomes, including cholesterol level, and the ED25 and the ED75 for each agent were tested at the 0.05 level without adjustment for multiple comparisons. Differences in smooth muscle content and VEGF protein expression from each treatment group were compared at the 0.05 level using the independent samples T-Test.

Results

Cholesterol levels. Serum cholesterol levels increased from 63.1±3.7 mg/dl to 1501.0±47.1 mg/dl after 7½ weeks on the 1% cholesterol diet (P<0.001). There as no difference in final cholesterol level between VEGF or vehicle-treated groups (1565.2±76.9 mg/dl, 1499.3±98.9 mg/dl, P=0.605).

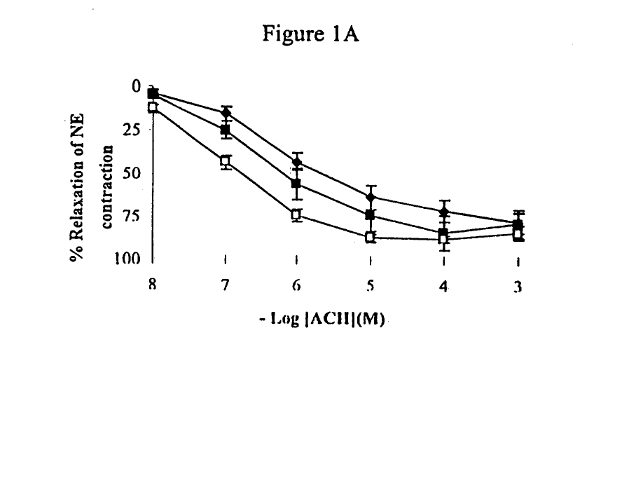

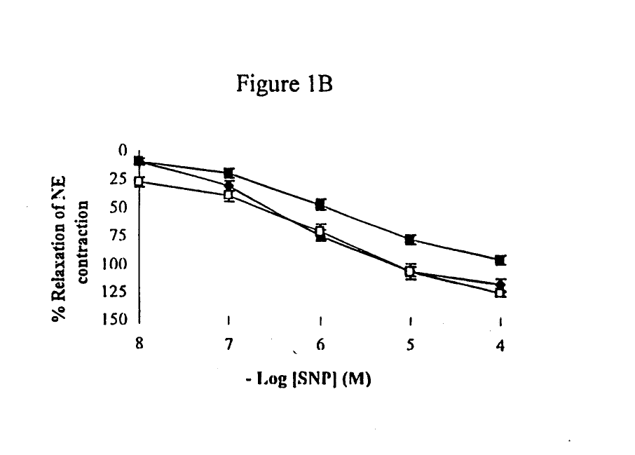

Isometric tension studies. The sensitivity to acetylcholine-mediated, endothelium-dependent relaxation was diminished in cholesterol-fed animals, as represented by the increase in ED50 (Table 1). Both the sensitivity and the maximal relaxation to sodium-nitroprusside-mediated, endothelium-independent relaxation were diminished in cholesterol-fed animals (Table 1). Endothelium-dependent smooth muscle relaxation was not affected by VEGF treatment (FIG. 1A). In the cholesterol-fed animals treated with either VEGF or VEGF vehicle, there was no significant difference in the sensitivity (ED50) to acetylcholine relaxation (4.94±0.25, 5.29±0.43, P=0.384) or the maximal relaxation to acetylcholine (78.6±6.2 grams, 87.8±9.2 grams. P=0.667). However, SNP-mediated, direct smooth muscle relaxation was significantly improved in cholesterol-fed, VEGF-treated rabbits (FIG. 1B). The sensitivity to sodium nitroprusside relaxation was not significantly different at ED50 (6.36±0.16, 6.00±0.18, P=0.148). However, the dose response curve was shifted to the left in the hypercholesterolemic VEGF-treated rabbits compared to hypercholesterolemic vehicle-treated animals, and this difference appears significant at ED75 (5.75±0.16, 5.23±0.18, P=0.046). Moreover, the maximal relaxation to sodium nitroprusside was significantly augmented in the hypercholesterolemic VEGF-treated animals compared to vehicle controls (113.9±6.3 grams, 95.2±3.5 grams, P=0.015).

TABLE 1
Isometric tension changes in the hypercholesterolemic
rabbit. In the cholesterol-fed, vehicle-treated animals,
endothelium-dependent (acetylcholine-ACH) and NO-mediated
(sodium nitroprusside-SNP), direct smooth muscle dysfunction
developed. with the sensitivity (ED50) to both agents
significantly decreased, and the maximal relaxation to sodium
nitroprusside significantly decreased.

| | ACH-ED50 | ACH-Percent | SNP-ED50 | SNP-Percent Maximal |
|---|---|---|---|---|
| Treatment Group | (−Log)(M) | Maximal Relaxation | (−Log)(M) | Relaxation |
| Cholesterol - Vehicle | 5.29 = 0.43 | 87.8 ± 9.2 | 6.00 ± 0.18 | 95.2 ± 3.5 |
| Normal diet - Vehicle | 6.50 = 0.19 | 92.8 ± 2.5 | 6.82 ± 0.16 | 125.1 ± 6.8 |
| Significance | P = 0.021 | P = 0.613 | P = 0.003 | P < 0.001 |

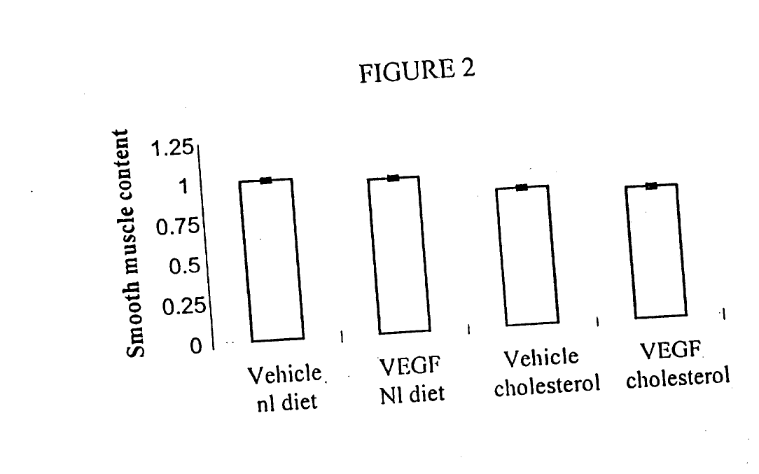

Trabecular smooth muscle content. Cholesterol-fed, vehicle-treated animals demonstrated significantly decreased overall smooth muscle content compared with normal diet controls (0.86±0.016 arbitrary units, 1±0.022 arbitrary units, P=0.008). Smooth muscle content did not differ among cholesterol-fed rabbits between vehicle or VEGF-treated groups (0.86±0.016 arbitrary units, 0.82±0.016 arbitrary units, P=0.450)(FIG. 2).

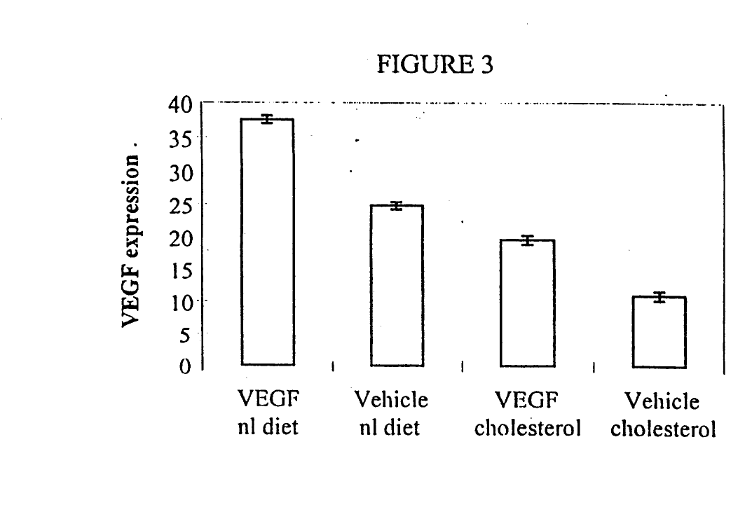

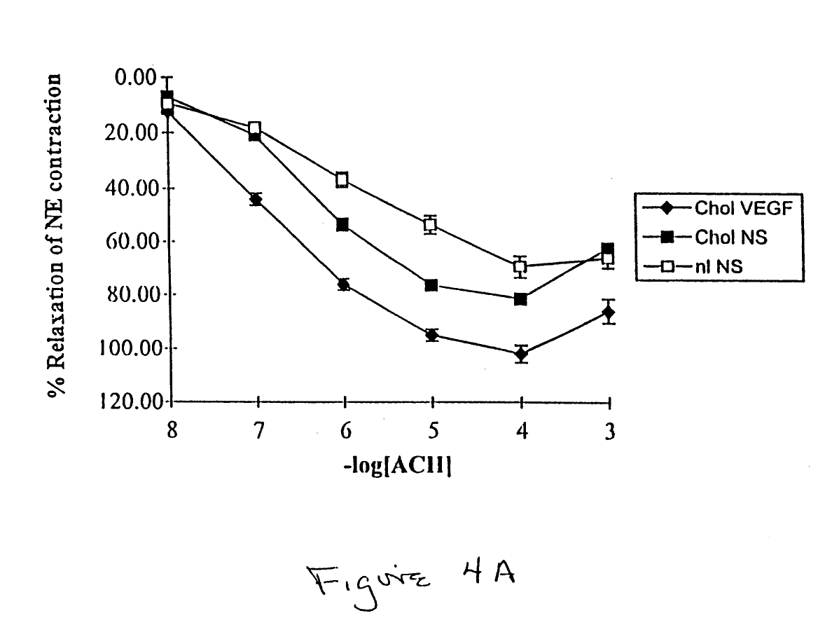

VEGF immunoexpression. Cholesterol-fed, vehicle-treated animals showed significantly decreased VEGF immunoexpression compared to normal diet controls (11.07±1.44 arbitrary units, 24.93±1.09 arbitrary units, P<0.001). In normal diet animals, VEGF-treated animals had higher VEGF expression than vehicle-treated animals (37.6±1.12 arbitrary units, 24.93±1.09 arbitrary units, P<0.001). In cholesterol fed animals, VEGF-treatment significantly augmented VEGF expression compared to vehicle-treated controls (19.67±1.38 arbitrary units. 11.07±1.44 arbitrary units, P<0.001) (FIG. 3).

### EXAMPLE 2

Intracavernosal Injections of VEGF Protect Endothelial Dependent Corporal Cavernosal Smooth Muscle Relaxation

Experimental Details

Fifteen male New Zealand White rabbits weighing 2 to 2.5 kg were used in a total of two arms in the study. There were five groups: (1) four rabbits were fed a 1% cholesterol diet (Harland Teklab, Wis.) for four weeks and received three (weekly) intracavernosal injections of saline; (2) four were fed a 1% cholesterol diet for four weeks and received three (weekly) intracavernosal injections of 0.3 mg VEGF; (3) three were given an intracavernosal injection of normal saline and fed standard rabbit chow (Purina Mills, Inc., St. Louis, Mo.) for four weeks; (4) three were given an intracavernosal injection of 1 mg of VEGF and fed standard rabbit chow for four weeks; and (5) one was fed standard rabbit chow with no injections and served as a control. Random serum total cholesterol levels were measured at the beginning of the study and after four weeks of the 1% cholesterol diet. At the end of four weeks, each rabbit underwent total penectomy and then was sacrificed. Each penis yielded two strips of tissue with a small amount of erectile tissue being placed in neutral buffered formalin 10%. The strips were suspended in tissue baths, and isometric tension studies were performed. Dose-response curves for norepinephrine were generated first in each strip to assess adrenergic-mediated cavernosal smooth muscle contraction. Next, dose-response curves for histamine were generated to assess histamine receptor mediated contraction of cavernosal smooth muscle. Submaximal contraction was then produced with 10⁻³ concentration of norepinephrine and dose-response curves were then generated to evaluate endothelial-dependent (acetylcholine) smooth muscle relaxation. Once again, a submaximal contraction was produced using 10⁻⁵ concentration of norepinephrine and dose response curves were then generated to evaluate endothelial-independent (sodium nitroprusside) smooth muscle relaxation.

Rabbit chow/feeding protocol. The custom 1% cholesterol diet consisted of 24,750 grams of standard rabbit chow, 250 grams cholesterol, and 50 grams calcium propionate per 25 kg barrel. Standard rabbit chow contains >2% fat, >14% protein, <20% fiber and <11% ash. Each rabbit was fed and consumed 120 grams of rabbit chow each day.

Tissue. Cavernosal tissue procurement was performed under general anesthesia induced with Ketamine 50 mg/kg SC (Ketaset, Bristol Laboratories, Syracuse, N.Y.) and Xylazine 30 mg/kg SC (Rompun, Mobay Corp., Shawnee, Kans.). The penis was excised en bloc and placed in warm Krebs solution, where the corpora cavernosa were sharply dissected from the tunica albuginea producing a strip (approximately 0.3×0.3×0.7 cm.) from each corpus. The strips were then mounted in the oxygen tissue baths. After tissue collection, the rabbits were euthanized with an overdose of intravenous sodium pentobarbitol (100 mg/kg to effect). Care was taken throughout the procedure to minimize tissue manipulation.

Tissue Chambers. Each cavernosal strip was placed in a 25 ml tissue bath (Kent Scientific Corp.. Litchfield, Conn.). One end was hooked to a tissue holder and the other end w as hooked to a force transducer (FTO3, Grass Instruments, Quincy, Mass.) for determination of isometric tension. The bath was filled with a modified Kreb's physiologic salt solution with the following millimolar composition: NaCl 122, KCl 4.7, MgCl₂ 1.2, CaCl₂ 2.5, NaHCO₃ 15.4, KH₂PO₄ 1.2. and glucose 5.5. A circulating water bath kept tissue chamber temperatures at 37C. Continuous aeration with 95% oxygen and 5% carbon dioxide maintained a pH of 7.4.

Optimal isometric tension determination. Each of the force transducers was connected to a transducer positioner enabling preload tension adjustment. Following suspension in the tissue chambers, the tension was periodically adjusted (at least every fifteen minutes) until the strip equilibrated at 0.5 gm. (usually two hours). The optimal preload tension was then determined by contracting the tissue with 60 mM KCl Krebs solution (prepared by substituting 60 mM of sodium with equimolar amounts of potassium in Krebs-PSS solution) at increasing levels of preload (0.5 gm. increments). Optimal preload tension was defined as that level of preload at which a further increase in tension failed to generate an increase of at least 10% in active tension: total tension minus resting tension. All subsequent testing was then performed at the determined optimal resting tension for each strip: Monitoring of tension was done with a four-channel polygraph (Grass 7D, Grass Instruments).

Isometric tension studies. The tissue was washed with Kreb's solution every fifteen minutes after each study until the tissue returned to baseline (at least one hour). Dose response curves for norepinephrine were obtained by cumulative addition of norepinephrine (10⁻⁹ to 10⁻⁴) in logarithmic increments. Next, dose response curves for histamine were obtained by cumulative addition of histamine (10⁻⁸ to 10⁻⁴ M) in logarithmic increments. Norepinephrine and histamine contractions are expressed as a percentage of maximal tension generated of each drug respectively. Each strip was then precontracted with 10⁻⁵ M of norepinephrine for assessment of relaxation by acetylcholine. Dose response curves were preformed by cumulative addition of acetylcholine (10⁻⁸ to 10⁻³ ) in logarithmic increments after steady-state contraction was attained. Lastly, 10⁻⁵ M of norepinephrine was used to precontract each strip for assessment of relaxation by sodium nitroprusside. Dose response curves were preformed by cumulative addition of sodium nitroprusside (10⁻⁸ to 10⁻⁴ M) in logarithmic increments after steady-state contraction was attained. Relaxation in response to acetylcholine and sodium nitroprusside is expressed as a percentage of the active tension generated by the 10⁻⁵ of norepinephrine.

Statistical analysis. Data are expressed as means±the standard error of the mean with n representing the number of cavernosal strips that were obtained. Dose response curves were compared by student's T-test. Statistical significance was considered when p<0.05. ED50, ED25, and ED75 were determined using logistic regression with logit transformation.

Results

Serum total cholesterol levels. There was a significant elevation in serum total cholesterol levels from a normal diet (38.7±5.53 mg./dl.) to after four weeks of a 1% cholesterol diet (727±75.6 mg./dl.) with p<0.01.

Intracavernosal Injection of VEGF. Intracavernosal injections of VEGF tended to produce an approximately 80% tumescence in the adult rabbit penis.

Isometric tension studies. There were no significant differences between the groups for the level of preload for optimal contraction or in the maximal active tension in response to 60 nM. KCl Krebs solution. Norepinephrine and histamine produced a dose-related contraction in all five groups. There was a slight increase in sensitivity to norepinephrine for the two cholesterol fed groups vs. the three normal diet groups. However the increase in sensitivity did not reach statistical significance (p>0.30): group 1 (fed a 1% cholesterol diet for four weeks and received three (weekly) intracavernosal injections of saline) ED₅₀=−5.29±0.13; group 2 (fed a 1% cholesterol diet for four weeks and received three (weekly) intracavernosal injections of 0.3 mg VEGF) ED₅₀=−5.34±0.17; group 3 (an intracavernosal injection of normal saline and fed standard rabbit chow for four weeks) ED₅₀=−5.29±0.2: group 4 (an intracavernosal injection of 1 mg of VEGF and fed standard rabbit chow for four weeks) ED₅₀=−5.11±0.14; and group 5 (fed standard rabbit chow with no injections) ED₅₀=−4.91±0.48. Histamine sensitivity by cholesterol also demonstrated a trend toward reduction, which did not reach statistical significance (p>0.18). Histamine sensitivity was reduced by cholesterol which was not affected by VEGF injections (p>0.4): group 1 ED₅₀=−5.19±0.27, group 2 ED₅₀=−5.19±0.1, group 3 ED₅₀=−0.05, group 4 ED₅₀=−5.45±0.11, and group 5 ED₅₀=−5.75±0.27. Both acetylcholine and sodium nitroprusside produced a dose related relaxation in all five groups. Acetylcholine sensitivity showed no significant differences: group 1 ED₅₀=−4.47±0.36, group 2 ED₅₀=−3.5735±0.61, group 3 ED₅₀=−3.76±0.22, and group 4 ED₅₀=−3.78±0.71. Nevertheless, acetylcholine showed a significant difference in the percent maximal relaxation between the hypercholesterolemic rabbits that received VEGF (94.5±8.41) as compared to the hypercholesterolemic rabbits that received NS (71.1±8.24) with p=0.033 (Table 2). The ED₅₀ of SNP showed no significant differences: group 1 ED₅₀=−6.48±0.16. group 2 ED₅₀=−6.09±0.16, group 3 ED₅₀=−5.344±0.52, group 4 ED₅₀=−5.87±0.21, and group 5 ED₅₀=−6.01±0.27. However, there was a significant difference in the relaxation to SNP of the hypercholesterolemic rabbits that received VEGF (−7.045±0.16) vs. NS (−6.65±0.14) at ED₂₅ with p=0.043. (Table 3).

TABLE 2
Dose-response relaxation of isolated strips of corpora
cavernosa from New Zealand White rabbits fed a 1% cholesterol
diet for four weeks and given three weekly intracavernosal
injections of either VEGF or NS to Acetylcholine.

| Sensitivity to | | | | Percent Maximal |
|---|---|---|---|---|
| Acetylcholine | ED²⁵ | ED⁵⁰ | ED⁷⁵ | Relaxation |
| Hypercholesterolemic | −5.48 ± 0.33 | −4.47 ± 0.36 | −3.50 ± 0.41 | 94.5 ± 8.41 |
| rabbits that received VEGF | | | | |
| Hypercholesterolemic | −4.74 ± 0.55 | −3.57 ± 0.61 | −2.37 ± 0.69 | 71.1 ± 8.24 |
| rabbits that received NS | | | | |
| P value | 0.132 | 0.115 | 0.092 | 0.033 |

TABLE 3
Dose-response relaxation of isolated strips of corpora
cavernosa from New Zealand White rabbits fed a 1% cholesterol
diet for four weeks and given three weekly intracavernosal
injections of either VEGF or NS to Sodium Nitroprusside.

| Sensitivity to Sodium | | | | Percent Maximal |
|---|---|---|---|---|
| Nitroprusside | ED²⁵ | ED⁵⁰ | ED⁷⁵ | Relaxation |
| Hypercholesterolemic | −6.65 ± 0.14 | −6.47 ± 0.16 | −5.52 ± 0.18 | 145 ± 8.26 |
| rabbits that received VEGF | | | | |
| Hypercholesterolemic | −6.02 ± 0.35 | −6.08 ± 0.16 | −4.66 ± 0.55 | 118 ± 6.87 |
| rabbits that received NS | | | | |
| P value | 0.043 | 0.055 | 0.068 | 0.159 |

### EXAMPLE 3

Intravenous VEGF Restores Corporal Smooth Muscle Relaxation

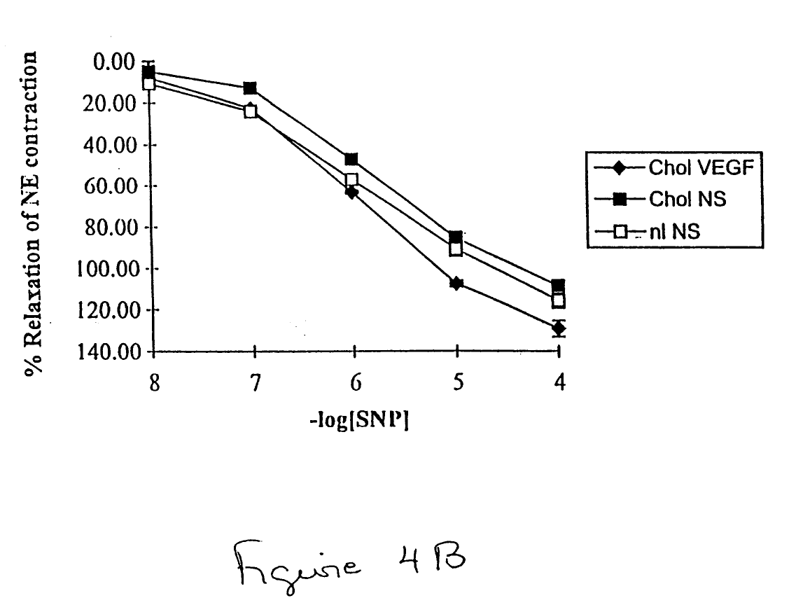

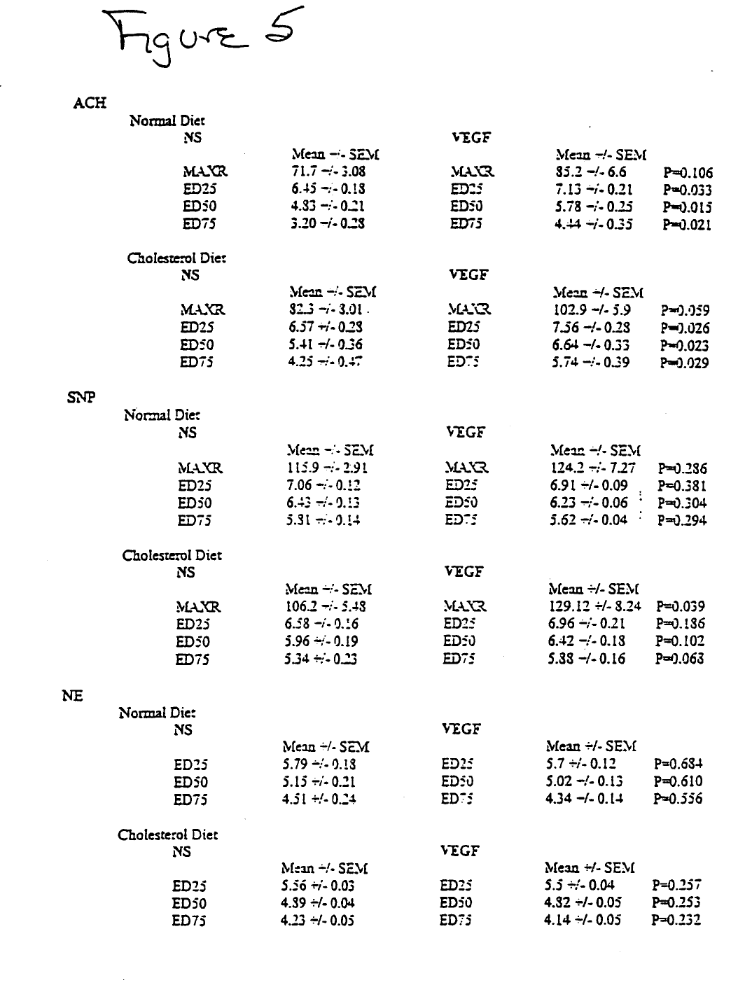

The route of administration can affect the efficacy of treatments for erectile dysfunction, as is seen in the difference between intrauretheral and intracavernosal alprostadil. A study was undertaken to determine the effects of intravenously delivered VEGF on both endothelial-dependent and endothelial-independent corporal smooth muscle relaxation in 12 New Zealand White rabbits fed a 1% cholesterol diet, who received a single intravenous bolus of either VEGF (0.9 mg) or VEGF-vehicle after 6 weeks. Ten days after injection isometric tension studies were performed on corporal tissue. Sensitivity and maximal relaxation to acetylcholine (ACH) and sodium nitroprusside (SNP) were compared between treatment groups. Sections of the corpora were assessed for smooth muscle content and for VEGF protein expression using immunohistochemistry. VEGF treatment significantly augmented endothelium dependent (ACH-mediated) (FIG. 4A) and endothelium independent (SNP-mediated) (FIG. 4B) maximal corporal smooth muscle relaxation (P=0.014, P=0.018). Moreover, the sensitivity (ED50) to both ACH and SNP was enhanced in the VEGF treated animals (P=0.004, P=0.001). VEGF protein immunoexpression was augmented after VEGF therapy (P=0.05). IV VEGF appears to restore both endothelial-dependent and endothelial-independent corporal smooth muscle function because it may allow more homogeneous application throughout the corpora than is achieved with IC injection.

### EXAMPLE 4

Intracorporal Vascular Endothelial Growth Factor Restores Corporal Smooth Muscle Response to Nitric Oxide

A study, was undertaken to determine if Vascular Endothelial Growth Factor (VEGF) could reverse the smooth muscle dysfunction in the hypercholesterolemic rabbit model of erectile dysfunction.

Twenty four New Zealand White rabbits were fed a 1% cholesterol diet or a normal diet, and received a single intracavernosal injection of either VEGF (0.9 mg) or VEGF-vehicle after 6 weeks. Ten days after injection, their corpora cavernosa were harvested, and isometric tension studies were performed. Relaxation to acetylcholine (ACH) and sodium nitroprusside (SNP) was compared within each group. Sections of the corpora were assessed for smooth muscle content with Masson Trichrome staining and for VEGF expression using immunohistochemistry.

Endothelium-dependent (ACH) and endothelium-independent (SNP) smooth muscle relaxation were both impaired in the cholesterol-fed animals (P=0.021, P=0.003). VEGF treatment restored the nitric oxide-mediated, direct smooth muscle relaxation to normal levels (P=0.015). Decreased smooth muscle content was found in cholesterol-fed animals versus normal diet controls (P=0.008), and this was not affected by VEGF treatment (P=0.450). VEGF expression was augmented after VEGF therapy (P<0.001).

In this rabbit model of hyperlipidemia-induced corporal smooth muscle injury, VEGF administration restores smooth muscle function to normal levels. Vasculogenic growth factors may have an important clinical role in the treatment of erectile dysfunction.

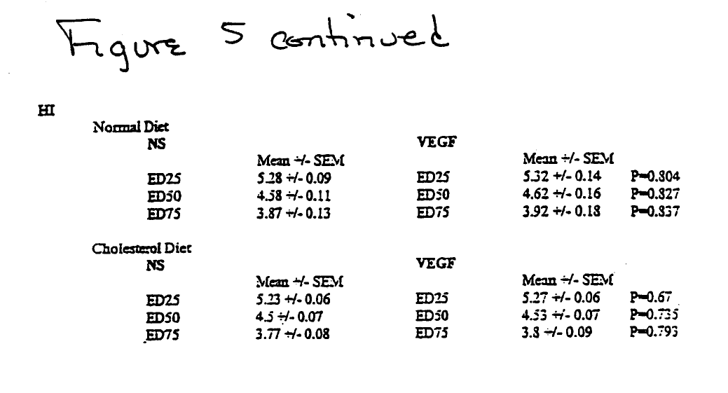

The above-described study was repeated and the results are set forth in FIG. 5.

### EXAMPLE 5

Duration of VEGF Effect in the Restoration of Corporal Vasoactive Function

24 NZW rabbits were given 0.9 mg of i.v. VEGF or 1 cc of VEGF vehicle after 6 weeks on a 1% cholesterol diet. 3 and 6 weeks after treatment, corporal tissue responses to acetylcholine (Ach) and sodium nitroprusside (Snp) were measured to gauge endothelium-dependent and independent responses, respectively. The effective dose to produce 25, 50, and 75% of maximum relaxation (ED25, 50, and 75) was calculated by log regression and expressed as the inverse log of dosage.

At 3 weeks, the Ach ED50 was significantly different for vehicle animals versus VEGF animals (3.8 vs. 4.9, p=0.01) with VEGF treated animals showing greater relaxation but the Snp ED50 did not demonstrate this effect (6.25 vs. 6.14, p=0.60). At 6 weeks the ED50 for Ach (4.4 vs. 5.5, p<0.01) and Snp (6.00 vs. 6.32. p<0.01) were significantly different with greater relaxation in the VEGF groups. ED25 and ED75 comparisons were consistent with these results.

VEGF treatment improves long term corporal vasoactive function after a single treatment. The duration of this effect is present at 6 weeks.

### EXAMPLE 6

VEGF Restores Corporal Smooth Muscle Function In Vitro

Experimental Details

Animals: 36 New Zealand White rabbits were fed either a normal rabbit diet (n=12) or a 1% cholesterol diet (n=24) (Harland Teklab, Madison, Wis.) for a total of 7.5 weeks. Twenty-four rabbits (half normal diet/half cholesterol diet) received an IC injection of either 0.9 mg recombinant VEGF-165 or an equivalent amount of VEGF-vehicle (Genentec, South San Francisco, Calif.) at week 6. Twelve cholesterol-fed rabbits received single IV injections of either 0.9 mg VEGF or VEGF-vehicle at week 6 (Table 4). Ten days after the injection, the rabbits were euthanized and underwent penectomy with meticulous dissection of the corpora cavernosa from the tunica albuginia. Total serum cholesterol was determined prior to the initiation of the experimental diet and immediately prior to the procedure.

TABLE 4
Treatment groups according to diet and therapy (# rabbits)

| | Agent and Route | | | |
|---|---|---|---|---|
| Animal Diet | IC Vehicle | IC VEGF | IV Vehicle | IV VEGF |
| 1% Cholesterol | 6 | 6 | 6 | 6 |
| Regular | 6 | 6 | | |

Isometric tension studies. As previously described (Kim et al, J. Urol. 151:198 (1994)), 2 corporal strips from each animal were suspended in 5 ml capacity organ baths containing Krebs physiological salt solution (112 mmol/liter. NaCl. 4.7 mmol/liter KCl, 1.2 mmol/liter MgCl₂, 2.5 mmol/liter CaCl₂, 15.4 mmol/liter NaHCO₃, 1.2 mmol/liter KH₂PO₄, and 5.5 mmol/liter glucose) maintained at 37C. and oxygenated with 95% O₂ and 5% CO₂. After equilibration at 0.5 grams, optimal preload tension was determined by contracting strips with 60 mmol KCl Krebs solution (60 mmol/liter NaCl, 1.2 mmol/liter MgCl₂, 2.5 mmol/liter CaCl₂, 15.4 mmol/liter NaHCO₃, 1.2 mmol/liter KH₂PO₄, and 5.5 mmol/liter glucose) at incrementally increasing levels of preload, until further increase in tension failed to generate an increase in active tension (total tension minus resting tension) of at least 10%. All subsequent testing was then performed at the optimal resting tension for each strip. Strips were sub-maximally pre-contracted with 10⁻⁵ M norepinephrine, and after a contractile plateau was reached, ACH (acetylcholine) (10⁻⁸ to 10⁻³ M) or SNP (sodium nitroprusside) (10⁻⁸ to 10⁻⁴ M) was added cumulatively in logarithmic increments. Endothelial dependent relaxation was assessed using ACH, while direct NO-mediated corporal smooth muscle dysfunction relaxation was assessed with SNP. Electrical field stimulation was not performed. Relaxation in response to each dose of either ACH or SNP is expressed as a percentage of the active tension generated by the 10⁻⁵ M dose of norepinephrine. These values were plotted against the negative logarithm of the agonist dose to produce relaxation dose-response curves. Logistic regression analysis with logit transformation was performed on the cumulative dose response curves from each treatment group to determine the ED25, ED50 and ED75 for each agent (Kim et al, J. Urol. 151:198 (1994); Finney, Statistical Method in Biological Assay, London: Charles Griffen & Co LDT (1978)). Treatment groups are compared at ED50 for ACH and SNP and at maximal relaxation to each agent.

Immunohistochemistry. Corporal sections were cryopreserved in 30% sucrose, snap frozen and sectioned (5 m). After acetone fixation for 10 minutes, phosphate-buffered saline wash and incubation with blocking solution (10% horse serum in phosphate-buffered saline), primary antibody was applied. The HHF35 monoclonal mouse antibody to f-actin incubated for 30 minutes was used for actin immunoexpression and smooth muscle quantification (Dako, Carpintera, Calif.). A monoclonal mouse antibody to CD-31 incubated overnight was used for CD-31 immunoexpression and endothelial quantification (Biogenics, Napa Calif.). A monoclonal horse anti-human VEGF antibody incubated for one hour was used for VEGF immunoexpression (Sigma, St. Louis, Mo.). The antigens were developed with the ABC reagent and the alkaline phosphatase substrate kit (Vector Labs, Burlingame, Calif.) with hematoxylin counterstaining, rendering antigen-expressing areas red. Ten randomly selected 40× fields per animal from each treatment group were analyzed using an image analysis system and overall smooth muscle area (actin) or endothelial area (CD-31) was quantified using NIH Image software. Likewise, ten randomly selected 40× VEGF-stained fields per animal from each treatment group were assessed, and areas of VEGF expression were counted by a single observer blinded to the treatment groups. The smooth muscle, endothelial, and VEGF-contents measured in normal diet, vehicle-treated rabbits were assigned a value of 100% (mean SEM), and other treatment groups were assessed relative to these values. IC and IV groups were assessed using the same protocol, but at different times with different antibody lots, and thus direct comparisons of staining patterns between these two routes may be biased.

ELISA evaluation of VEGF expression. Frozen corporal tissues were sonicated in 50 mM Tris radioimmunoprecipation assay buffer for 1 minute for protein isolation. Protein concentrations were determined using the Bradford protein assay, and then 60 ug of protein from each sample was boiled for 5 minutes prior to analysis using a VEGF ELISA kit (Quantikine, R&D Systems, Minneapolis, Minn.). The samples were quantified with a Molecular Devices Kinetic microplate reader using Apple SoftMax software. One ELISA was performed for each animal in each treatment group.

Statistical evaluation. Data are expressed as the mean standard error of the mean (SEM). The responses of strips from each treatment group to ACH or SNP were compared using the independent samples T-Test after samples were checked for normality and no significant deviations were found. Statistical significance was determined at the 0.05 level.

Results

Cholesterol levels. Serum cholesterol levels increased from 63.1 3.7 mg/dl to 1501.0 47.1 mg/dl after 7½ weeks on the 1% cholesterol diet (P<0.001). There was no difference in final cholesterol level between VEGF and vehicle-treated groups (1565.2 76.9 mg/dl. 1499.3 98.9 mg/dl, P=0.605). The final serum cholesterol level in control animals was 60.1 1.4 mg/dl, not significantly changed from the initial cholesterol level.

Isometric tension studies. The ED50 (-Log (M)) to ACH-mediated, endothelium-dependent relaxation was diminished in cholesterol-fed animals compared to normal controls (Table 5). Both the ED50 and the maximal relaxation to direct NO-mediated (SNP) relaxation were diminished in cholesterol-fed animals compared to normal diet animals (Table 6).

TABLE 5
Acetylcholine isometric tension studies

| | ED50 (−Log (M)) | Significance | Maximal Relaxation | Significance |
|---|---|---|---|---|
| Cholesterol / IC Vehicle | 5.29 0.43 | P = 0.021 | 87.8% 9.2% | P = 0.613 |
| Normal diet / IC Vehicle | 6.50 0.19 | | 92.8% 2.5% | |
| Cholesterol / IC VEGF | 4.94 0.25 | P = 0.384 | 78.6% 6.2% | P = 0.667 |
| Cholesterol / IC Vehicle | 5.29 0.43 | | 87.8% 9.2% | |
| Cholesterol / IV VEGF | 6.33 0.40 | P = 0.004 | 96.1% 8.9% | P = 0.014 |
| Cholesterol / IV Vehicle | 4.83 0.25 | | 71.6% 4.0% | |

TABLE 6
Sodium Nitroprusside isometric tension studies

| | ED50 (−Log (M)) | Significance | Maximal Relaxation | Significance |
|---|---|---|---|---|
| Cholesterol / IC Vehicle | 6.00 0.18 | P = 0.003 | 95.2% 3.5% | P < 0.001 |
| Normal diet / IC Vehicle | 6.82 0.16 | | 125.1% 6.8% | |
| Cholesterol / IC VEGF | 6.36 0.16 | P = 0.148 | 113.9% 6.3% | P = 0.015 |
| Cholesterol / IC Vehicle | 6.00 0.18 | | 95.2% 3.5%, | |
| Cholesterol / IV VEGF | 6.93 0.25 | P = 0.001 | 116.6% 7.8% | P = 0.018 |
| Cholesterol / IV Vehicle | 5.70 0.13 | | 95.6% 3.6% | |

Endothelium-dependent smooth muscle relaxation was not affected by IC VEGF treatment (Table 5). In the cholesterol-fed animals treated with either IC VEGF or vehicle, there was no significant difference in the ED50 to ACH relaxation or the maximal relaxation to ACH. However, NO-mediated, direct smooth muscle relaxation was significantly improved in cholesterol-fed, IC VEGF-treated rabbits (Table 6). While the SNP relaxation was not significantly different at ED50, the dose response curve was shifted to the left in the VEGF-treated rabbits and this difference may be significant at ED75 (5.75 0.16, 5.23 0.18, P=0.046). Moreover, the maximal relaxation to SNP was significantly augmented in the IC VEGF-treated animals compared to vehicle controls. Endothelium-dependent smooth muscle relaxation was augmented by IV VEGF treatment (Table 5), with a significant difference in the ED50 to ACH relaxation and the maximal relaxation to ACH. Likewise, SNP-mediated, direct smooth muscle relaxation was significantly improved in cholesterol-fed, IV VEGF-treated rabbits (Table 6) at ED50 and maximal relaxation.

Endothelial cell content. As measured by CD-31 staining, endothelial cell content was significantly augmented in normal diet animals after IC VEGF versus vehicle (136% 6%, 100% 5%, P 0.001). In cholesterol-fed animals, endothelial cell content was increased after IC VEGF versus vehicle (114% 6%, 81% 6%, P=0.006). After IV VEGF versus vehicle however this difference was not evident (83% 4%, 87% 5%, P=0.385).

Trabecular smooth muscle content. Cholesterol diet, vehicle-treated animals demonstrate significantly decreased overall smooth muscle content by actin staining compared to normal diet controls (56% 3%, 100% 3%, P=<0.001). Smooth muscle content did not differ among cholesterol-fed rabbits between IC VEGF and vehicle-treated groups (63% 2%, 56% 3%, P=0.087). Likewise, actin staining was not significantly different between IV VEGF and IV vehicle groups (41% 2%, 46% 3%, P=0.102).

VEGF immunoexpression. Cholesterol diet, vehicle-treated animals demonstrated significantly decreased VEGF immunoexpression compared to normal diet controls (44% 6%, 100% 4%, P<0.001). In normal diet animals, VEGF-treated animals had higher VEGF expression than vehicle-treated animals (151% 4%, 100% 4%, P<0.001). In cholesterol fed animals, IC VEGF-treatment significantly augmented VEGF expression compared to vehicle-treated controls (79% 6%, 44% 6%, P<0.001)(FIG. 5). Likewise, IV VEGF augmented VEGF immunoexpression in cholesterol-fed animals versus vehicle (P=0.051).

VEGF ELISA. Optical density (OD) minus background in cholesterol-fed animals treated with VEGF was greater than that in vehicle-treated animals, although this did not reach statistical significance (0.168, 0.094, P=0.086).

Likewise, OD was increased after IV VEGF administration compared to vehicle (0.128. 0.91, P=0.519).

All documents cited above are herebv incorporated in their entirety by reference.

One skilled in the art will appreciate from a reading of this disclosure that vanious changes in form and detail can be made without departing from the true scope of the invention.

What is claimed is:

1. A method of preventing or treating dysfunction of penile, clitoral or vaginal erectile tissue comprising administering to a patient in need thereof an effective amount of an angiooenic growth factor, or active fragment thereof or mimetic thereof.

2. The method according to claim 1 wherein said patient is male.

3. The method according to claim 1 wherein said angiogenic growth factor is VEGF or FGF.

4. The method according to claim 1 further comprising administering an endothelial growth factor.

5. The method according to claim 4 wherein said endothelial growth factor is angiopoietin I.

6. The method according to claim 1 wherein said angiogenic growth factor is administered intravenously.

7. The method according to claim 6 wherein said angiogenic growth factor is administered intracavernosally.

8. A method of treating erectile dysfunction comprising administering to a male patient in need thereof an effective amount of an angiogenic growth factor, or active fragment thereof or mimetic thereof.

9. The method according to claim 8 wherein said angiogenic growth factor is VEGF or FGF.

10. The method according to claim 8 wherein said angiogenic growth factor is administered intracavernosally.
